(19) United States
(12) Patent Application Publication
(10) Pub. No.: US 2021/0198248 A1
(43) Pub. Date: Jul. 1, 2021

(54) FUMARATE SALT OF 5-((5-METHYL-2-((3,4,5-TRIMETHYLPHENYL)AMINO)PYRIMIDIN-4-YL)AMINO)-BENZO[D]OXAZOL-2(3H)-ONE

(71) Applicant: ASTRAZENECA AB, Sodertalje (SE)

(72) Inventors: Håkan SCHULZ, Sodetalje (SE); Reed Warren SMITH, JR., Sodertalje (SE)

(21) Appl. No.: 17/057,983

(22) PCT Filed: May 20, 2019
(86) PCT No.: PCT/EP2019/062935

§ 371 Date: Nov. 23, 2020

Related U.S. Application Data
(60) Provisional application No. 62/675,820, filed on May 24, 2018.

(51) Int. Cl. C07D 413/12

(52) U.S. Cl. CPC C07D 413/12; C07B 2200/13

(57) ABSTRACT

A fumarate salt, in particular the hemi-fumarate salt, of 5-((5-methyl-2-((3,4,5-trimethylphenyl)amino)pyrimidin-4-yl)amino)-benzo[d]oxazol-2(3H)-one (Compound (I), compositions comprising such a salt, and processes for the manufacture of such a salt, in particular Compound (I) hemi-fumarate salt are described. The salt is useful for the treatment of conditions such as asthma and COPD, involving modulation of the JAK pathway or inhibition of JAK kinases particularly JAK1.

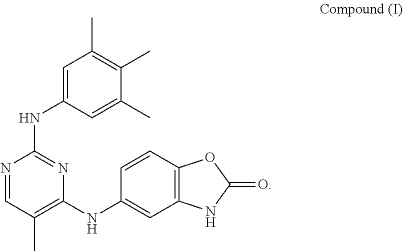 [SMILES: Cc1cnc(Nc2cc(C)c(C)c(C)c2)nc1Nc1ccc2oc(=O)[nH]c2c1]

The present disclosure relates to a salt of 5-((5-methyl-2-((3,4,5-trimethylphenyl)-amino)pyrimidin-4-yl)amino)benzo[d]oxazol-2(3H)-one hereafter “Compound (I)”, more particularly to a fumarate salt of Compound (I).

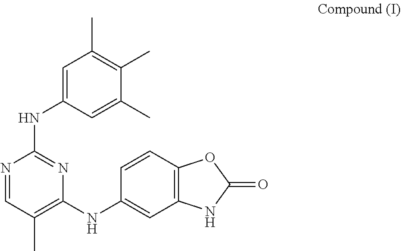 [SMILES: Cc1cnc(Nc2cc(C)c(C)c(C)c2)nc1Nc1ccc2oc(=O)[nH]c2c1]

The fumarate salt is expected to be useful for the treatment or prophylaxis of conditions mediated alone or in part by JAnus Kinases (or JAK) which are a family of cytoplasmic protein tyrosine kinases including JAK1, JAK2, JAK3 and TYK2. Each of the JAK kinases is selective for the receptors of certain cytokines, though multiple JAK kinases can be affected by particular cytokine or signaling pathways. Studies suggest that JAK3 associates with the common gamma chain (γc) of the various cytokine receptors. In particular, JAK3 selectively binds to receptors and is part of the cytokine signalling pathway for IL-2, IL-4, IL-7, IL-9, IL-15 and IL-21. The kinase JAK1 interacts with, among others, the receptors for cytokines IL-2, IL-4, IL-7, IL-9 and IL-21. Upon the binding of certain cytokines to their receptors (e.g., IL-2, IL-4, IL-7, IL-9, IL-15 and IL-21), receptor oligomerization occurs, resulting in the cytoplasmic tails of associated JAK kinases being brought into proximity and facilitating the trans-phosphorylation of tyrosine residues on the JAK kinase. This trans-phosphorylation results in the activation of the JAK kinase.

Phosphorylated JAK kinases bind various Signal Transducer and Activator of Transcription (STAT) proteins. These STAT proteins, which are DNA binding proteins activated by phosphorylation of tyrosine residues, function both as signaling molecules and transcription factors and ultimately bind to specific DNA sequences present in the promoters of cytokine-responsive genes (Leonard et al., (2000), J. Allergy Clin. Immunol. 105:877-888). Signaling of JAK/STAT has been implicated in the mediation of many abnormal immune responses such as allergies, asthma, autoimmune diseases such as transplant (allograft) rejection, rheumatoid arthritis, amyotrophic lateral sclerosis and multiple sclerosis, as well as in solid and hematologic malignancies such as leukemia and lymphomas. For a review of the pharmaceutical intervention of the JAK/STAT pathway see Frank, (1999), Mol. Med. 5:432:456 and Seidel et al., (2000), Oncogene 19:2645-2656 and Vijayakriishnan et al, Trends Pharmacol. Sci 2011, 32, 25-34 and Flanagan et al, J. Med. Chem. 2014, 57, 5023-5038.

Given the importance of JAK kinases compounds which modulate the JAK pathway can be useful for treating diseases or conditions where the function of lymphocytes, macrophages, or mast cells is involved (Kudlacz et al., (2004) Am. J. Transplant 4:51-57; Changelian (2003) Science 302:875-878). Conditions in which targeting of the JAK pathway or modulation of the JAK kinases are contemplated to be therapeutically useful include, leukemia, lymphoma, transplant rejection (e.g., pancreas islet transplant rejection, bone marrow transplant applications (e.g., graft-versus-host disease), autoimmune diseases (e.g., diabetes), and inflammation (e.g., asthma, allergic reactions).

In view of the numerous conditions that are contemplated to benefit by treatment involving modulation of the JAK pathway it is apparent that new compounds and new forms of compounds that modulate JAK pathways and methods of using these compounds should provide substantial therapeutic benefits to a wide variety of patients.

Compound (I) is described in International Patent Application WO 2010/085684, disclosing a genus of JAK inhibiting compounds and 700+ specific compounds (including N2-(3,4,5-trimethyl)phenyl-5-methyl-N4-(2-oxo-2,3-dihydro-1,3-benzoxazol-5-yl)-2,4-pyrimidinediamine in free base form—see Example I-365). N2-(3,4,5-trimethyl)-phenyl-5-methyl-N4-(2-oxo-2,3-dihydro-1,3-benzoxazol-5-yl)-2,4-pyrimidinediamine may also be named as 5-((5-methyl-2-((3,4,5-trimethylphenyl)amino)pyrimidin-4-yl)amino)-benzo[d]oxazol-2(3H)-one. International Patent Application WO 2012/15972 describes approximately 250 additional JAK inhibiting compounds, including various salts of N2-(3,4,5-trimethyl)phenyl-5-methyl-N4-(2-oxo-2,3-dihydro-1,3-benzoxazol-5-yl)-2,4-pyrimidinediamine. There is no description in WO 2010/085684 or WO 2012/15972 of a salt with fumaric acid of Compound (I).

We have now found that Compound (I) can be prepared as a fumarate salt, in particular as a hemi-fumarate salt, useful in the treatment of conditions in which targeting of the JAK pathway or inhibition of JAK kinases, particularly JAK1, are therapeutically useful.

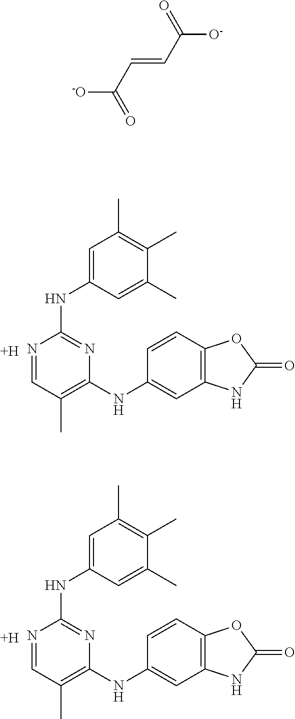

### 5-((5-methyl-2-((3,4,5-trimethylphenyl)-amino)pyrimidin-4-yl)amino)benzo[d]oxazol-2(3H)-one hemi-fumarate Salt

Compound (I) hemi-fumarate salt has a Compound (I):fumaric acid stoichiometry of 1:2 (as shown above). Other Compound (I) fumarate salt stoichiometries are possible, for example a Compound (I):fumaric acid ratio of 1:1 and it is to be understood that the disclosure encompasses all such stoichiometries of Compound (I):fumaric acid.

We have found that the hemi-fumarate salt, in particular, of Compound (I) has favourable properties compared to Compound (I) free base. For example, Compound (I) hemi-fumarate salt has a favourable dissolution profile exhibiting, high aqueous solubility and a good intrinsic dissolution rate.

According to a first aspect of the present disclosure there is provided Compound (I) fumarate salt, in particular the hemi-fumarate salt of Compound (I).

Suitably the Compound (I) hemi-fumarate salt is crystalline. According to a further aspect of the present disclosure there is provided crystalline Compound (I) hemi-fumarate salt.

The Compound (I) fumarate salt, in particular the Compound (I) hemi-fumarate salt, may exist in solvated as well as unsolvated forms such as, for example, hydrated forms. It is to be understood that the disclosure encompasses all such solvated and unsolvated forms of Compound (I) fumarate salt, in particular of the Compound (I) hemi-fumarate salt.

We have found that a particular crystalline form of Compound (I) hemi-fumarate salt, hereafter “Form A”, is characterised in that it provides an X-ray powder diffraction (XRPD) pattern substantially as shown in FIG. 1. The most prominent peaks of Form A are shown in Table 1 (see Example 1).

According to a further aspect of the disclosure there is provided Form A, wherein said Form A has an X-ray powder diffraction pattern with specific peaks at about 11.3, 16.9, 27.2 °2θ.

According to a further aspect of the disclosure there is provided Form A, wherein said Form A has an X-ray powder diffraction pattern with specific peaks at about 11.3, 14.5, 16.9, 22.6, 27.2 °2θ.

According to another aspect of the disclosure there is provided Form A, wherein said Form A has an X-ray powder diffraction pattern substantially the same as the X-ray powder diffraction pattern shown in FIG. 1.

Differential scanning calorimetry (FIG. 2) on Compound (I) hemi-fumarate salt shows an endotherm melting with an onset temperature of 307° C.

Suitably, Form A is substantially free of other forms of Compound (I) hemi-fumarate salt. For example, at least 80% of the Compound (I) hemi-fumarate salt is in the form of Form A, particularly at least 90%, more particularly, at least 95% and still more particularly at least 99% of the Compound (I) hemi-fumarate salt is in the form of Form A. In a particular embodiment at least 98% of the Compound (I) hemi-fumarate salt is in the form of Form A. Reference herein to, for example, 80% of the Compound (I) hemi-fumarate salt being in the form of Form A, refer to the % by weight of the Compound (I) hemi-fumarate salt.

The Compound (I) hemi-fumarate salt described herein is crystalline. Suitably the degree of crystallinity as determined by X-ray powder diffraction data is for example greater than about 60%, such as greater than about 80%, particularly greater than about 90% and more particularly greater than about 95%. In embodiments of the disclosure, the degree of crystallinity as determined by X-ray powder diffraction data is greater than about 98%, wherein the % crystallinity refers to the % by weight of the total sample mass which is crystalline.

In the preceding paragraphs & claims defining the X-ray powder diffraction peaks for the crystalline forms of Compound (I), the term “at about” is used to indicate that the precise position of peaks (i.e. the recited 2-theta angle values) should not be construed as being absolute values because, as will be appreciated by those skilled in the art, the precise position of the peaks may vary slightly between one measurement apparatus and another, from one sample to another, or as a result of slight variations in measurement conditions utilised. It is also stated in the preceding paragraphs that the Compound (I) hemi-fumarate salt Form A provides X-ray powder diffraction patterns ‘substantially’ the same as the X-ray powder diffraction patterns shown in FIG. 1, and has substantially the most prominent peaks (2-theta angle values) shown in Table 1. It is to be understood that the use of the term ‘substantially’ in this context is also intended to indicate that the 2-theta angle values of the X-ray powder diffraction patterns may vary slightly from one apparatus to another, from one sample to another, or as a result of slight variations in measurement conditions utilised, so the peak positions shown in the Figure or quoted are again not to be construed as absolute values.

It is known in the art that an X-ray powder diffraction pattern may be obtained which has one or more measurement errors depending on measurement conditions (such as equipment, sample preparation or machine used). In particular, it is generally known that intensities in an X-ray powder diffraction pattern may fluctuate depending on measurement conditions and sample preparation. For example, persons skilled in the art of X-ray powder diffraction will realise that the relative intensities of peaks may vary according to the orientation of the sample under test and on the type and setting of the instrument used. The skilled person will also realise that the position of reflections can be affected by the precise height at which the sample sits in the diffractometer and the zero calibration of the diffractometer. The surface planarity of the sample may also have a small effect. Hence a person skilled in the art will appreciate that the diffraction pattern data presented herein is not to be construed as absolute and any crystalline form that provides a power diffraction pattern substantially identical to those disclosed herein fall within the scope of the present disclosure (for further information see Jenkins, R & Snyder, R. L. ‘Introduction to X-Ray Powder Diffractometry’ John Wiley & Sons, 1996).

Generally, a measurement error of a diffraction angle in an X-ray powder diffractogram may be approximately plus or minus 0.1° 2-theta, and such a degree of a measurement error (i.e. plus or minus 0.1°) should be taken into account when considering the X-ray powder diffraction data herein. Furthermore, it should be understood that intensities might fluctuate depending on experimental conditions and sample preparation (e.g. preferred orientation).

It is known that the melting point onset temperature may be affected by several parameters such as impurity content, particle size, sample preparation and the measurement conditions (e.g. heating rate). It will be appreciated that alternative readings of melting point may be given by other types of equipment or by using conditions different to those described hereinar. Hence the melting point and endotherm figures quoted herein are not to be taken as absolute values and such measurement errors are to be taken into account when interpreting DSC data. Typically, melting points may vary by ±0.5° C. or less.

The crystalline form of Compound (I) hemi-fumarate salt, such as Form A according to the present disclosure may also be characterised and/or distinguished from other physical forms using other suitable analytical techniques, for example NIR spectroscopy or solid state nuclear magnetic resonance spectroscopy.

The chemical structure of Compound (I) fumarate salt, in particular of the Compound (I) hemi-fumarate salt, of the present disclosure can be confirmed by routine methods for example proton nuclear magnetic resonance (NMR) analysis.

### Synthesis of Compound (I) Free Base

Compound (I) may be synthesised using the methods described in WO 2010/085684 or as illustrated in the Examples herein.

Compound (I) free base has also been prepared according to the process illustrated in Reaction Scheme 1, in which Intermediate 1 is charged to a reactor with methanol followed by sodium bicarbonate and water, and reacted with Intermediate 2.

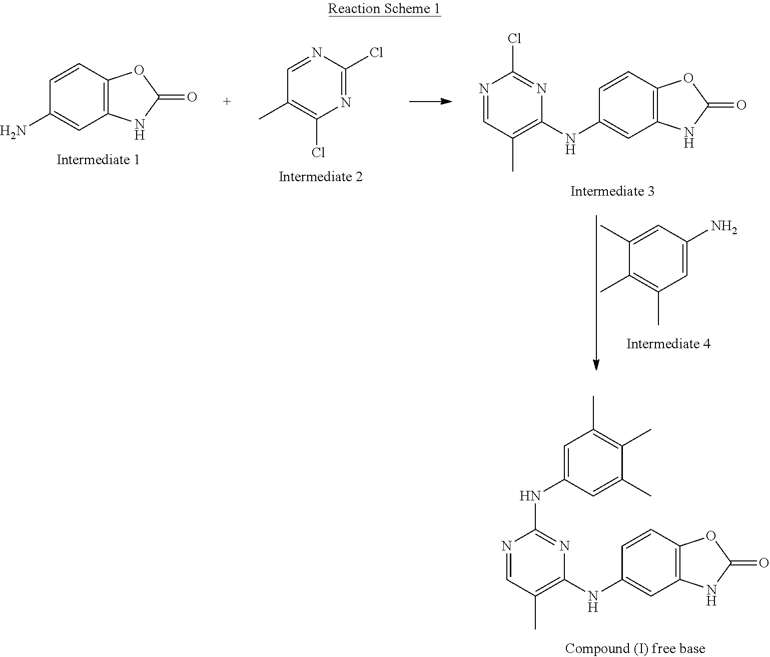

Intermediates 3 and 4 are reacted as described in the Examples.

Further, re-crystallisation of Compound (I) free base from certain solvents, such as DMSO, provides Compound (I) in high purity. Furthermore, dissolution of Compound (I) free base in DMSO provides a process, as outlined below, for preparation of Compound (I) hemi-fumarate salt, which may be suitable for large-scale manufacture of Compound (I) hemi-fumarate salt.

### Synthesis of Compound (I) Fumarate Salt, in Particular Compound (I) Hemi-Fumarate Salt

According to a further aspect of the present disclosure there is provided a process for the preparation of Compound (I) fumarate salt, in particular of the Compound (I) hemi-fumarate salt comprising:
(i) Dissolving Compound (I) free base in a suitable solvent;
(ii) Dissolving fumaric acid a suitable solvent;
(iii) Mixing the two solutions;
(iv) Optionally adding seed crystals of Compound (I) (hemi-)fumarate salt;
(v) Optionally adding an anti-solvent, such as methanol or ethanol;
(vi) Crystallising the Compound (I) (hemi-)fumarate salt;
(vii) Optionally washing the crystals with a solvent, such as water and/or methanol; and
(viii) Isolating Compound (I) (hemi-)fumarate salt.

### Notes on Steps (i) and (ii)

Conveniently Compound (I) free base is dissolved in a suitable solvent, such as DMSO (dimethyl sulfoxide). Conveniently the fumaric acid is dissolved in a suitable solvent, such as DMSO.

Crystallisation may be effected using known methods for crystallisation of a compound from solution, for example by adding seed crystals or by causing supersaturation of the solution containing the (hemi-)fumarate salt. Supersaturation may be achieved by, for example, cooling the solution, evaporating solvent from the solution or by addition of a suitable anti-solvent to the solution.

Crystalline Compound (I) hemi-fumarate salt may be prepared by, for example, the methods described herein in the Examples. The product obtainable by any of the processes of the specification and/or the Examples is a further aspect of the disclosure.

### Pharmaceutical Compositions

Compound (I) fumarate salt, in particular the Compound (I) hemi-fumarate salt, may be administered by inhalation as miconised solid particles without any additional excipients, diluents or carriers. Compound (I) fumarate salt, in particular the Compound (I) hemi-fumarate salt, may also be administered in a suitable pharmaceutical composition.

According to a further aspect of the disclosure there is provided a pharmaceutical composition which comprises Compound (I) fumarate salt, in particular the Compound (I) hemi-fumarate salt, in association with a pharmaceutically-acceptable diluent or carrier. The Compound (I) hemi-fumarate salt may be used in the composition in any form described herein, for example Form A.

The compositions of the disclosure may be in a form suitable for administration by inhalation (for example as a finely divided powder or a liquid aerosol) or for administration by insufflation (for example as a finely divided powder) using a suitable device.

The compositions of the disclosure may be obtained by conventional procedures using conventional pharmaceutical excipients, well known in the art. Thus, compositions intended for inhalation may contain, for example, micronized lactose or other suitable excipients, for example in an amount up to 90 w/w % of the composition.

If required, Compound (I) fumarate salt, in particular the Compound (I) hemi-fumarate salt, may be milled or micronized prior to formulation to provide a uniform particle size distribution of the Compound (I) hemi-fumarate salt. For example the Compound (I) hemi-fumarate salt may be milled to provide an average particle size of about 1 μm to 3 μm. Suitable milling and micronisation methods are well known.

The amount of active ingredient that is combined with one or more excipients to produce a single dosage form will necessarily vary depending upon the host treated and the particular route of administration. For example, a formulation intended for inhalation in humans will generally contain, for example, from approximately 0.005 mg to 10 mg of active agent compounded with an appropriate and convenient amount of excipient/s, which may vary from about 5 to about 95 percent by weight of the total composition.

The size of the dose for therapeutic or prophylactic purposes of Compound (I) fumarate salt, in particular the Compound (I) hemi-fumarate salt, will naturally vary according to the nature and severity of the conditions, the age and sex of the animal or patient and the route of administration, according to well known principles of medicine.

For administration by inhalation, a dose in the range, for example, 0.1 μg/kg to 0.1 mg/kg body weight will typically be used, for example 5 μg/kg.

The Compound (I) fumarate salt, in particular the Compound (I) hemi-fumarate salt, dissociates in aqueous media to the free base Compound (I) which has the biological activity as assessed in the tests & assays described in WO 2010/085684 (see, for example, page 314 showing that in a cell-based assay, Example I-365 has JAK activity IC₅₀<0.5 μM).

Accordingly, the Compound (I) fumarate salt, in particular the Compound (I) hemi-fumarate salt, of the present disclosure is expected to be useful in the treatment of diseases or medical conditions mediated alone or in part by JAK, particularly JAK1, i.e. Compound (I) fumarate salt, in particular the Compound (I) hemi-fumarate salt, may be used to produce a JAK-inhibitory effect in a warm-blooded animal in need of such treatment.

Importantly, the Compound (I) fumarate salt, in particular the Compound (I) hemi-fumarate salt, of the present disclosure can be used to inhibit JAK kinases in vivo as a therapeutic approach towards the treatment or prevention of diseases mediated, either wholly or in part, by a JAK kinase activity (referred to herein as “JAK kinase mediated diseases”). Non-limiting examples of JAK kinase mediated diseases that can be treated or prevented include those mentioned in WO 10/085684 such as allergies and asthma.

In addition to the disorders listed above, Compound (I) fumarate salt, in particular the Compound (I) hemi-fumarate salt, can be useful for the treatment of obstructive, restrictive or inflammatory airways diseases of whatever type, etiology, or pathogenesis, in particular an obstructive, restrictive or inflammatory airways disease, including, as mentioned above, asthma, in particular atopic asthma, allergic asthma, non-atopic asthma, bronchial asthma, non-allergic asthma, emphysematous asthma, exercise-induced asthma, emotion-induced asthma, extrinsic asthma caused by environmental factors, infective asthma associated with bacterial, fungal, protozoal and/or viral infection, bronchiolitis, cough variant asthma, drug induced asthma, and the like, rhinitis or sinusitis of different etiologies, including without limitation, seasonal allergic rhinitis, perennial allergic rhinitis, vasomotor rhinitis, sinusitis, including acute, chronic, ethmoid, frontal maxillary or sphenoid sinusitis; chronic obstructive pulmonary disease (COPD), chronic obstructive lung disease (COLD), chronic obstructive airways disease (COAD) or small airways obstruction, including, without limitation, chronic bronchitis, pulmonary emphysema, bronchiectasis, cystic fibrosis, bronchiolitis obliterans; bronchitis, including in particular, acute bronchitis, acute laryngotracheal bronchitis, chronic bronchitis, dry bronchitis, productive bronchitis, infectious asthmatic bronchitis, staphylococcus or streptococcal bronchitis and vesicular bronchitis.

Accordingly, there is provided a Compound (I) fumarate salt, in particular the Compound (I) hemi-fumarate salt, for use as a medicament.

According to a further aspect there is provided a Compound (I) fumarate salt, in particular the Compound (I) hemi-fumarate salt, for use in the production of a JAK-inhibitory effect in a warm-blooded animal such as man.

Thus according to this aspect there is provided the use of a Compound (I) fumarate salt, in particular the Compound (I) hemi-fumarate salt, in the manufacture of a medicament for use in the production of a JAK-inhibitory effect in a warm-blooded animal such as man.

According to a further feature of this aspect there is provided a method for producing a JAK-inhibitory effect in a warm-blooded animal, such as man, in need of such treatment which comprises administering to said animal an effective amount of a Compound (I) fumarate salt, in particular the Compound (I) hemi-fumarate salt.

According to a further aspect there is provided a Compound (I) fumarate salt, in particular the Compound (I) hemi-fumarate salt, for use in the prevention or treatment of asthma or COPD.

According to a further aspect there is provided the use of a Compound (I) fumarate salt, in particular the Compound (I) hemi-fumarate salt in the manufacture of a medicament for use in the prevention or treatment of asthma or COPD.

According to a further feature of this aspect there is provided a method for preventing or treating asthma or COPD in a warm-blooded animal, such as man, in need of such treatment which comprises administering to said animal an effective amount of a Compound (I) fumarate salt, in particular the Compound (I) hemi-fumarate salt.

The Compound (I) fumarate salt, in particular the Compound (I) hemi-fumarate salt, of the present disclosure may be used in combination with other active ingredients by simultaneous, separate or sequential administration. In one aspect of the disclosure “combination” refers to simultaneous administration. In another aspect of the disclosure “combination” refers to separate administration. In a further aspect of the disclosure “combination” refers to sequential administration. Where the administration is sequential or separate, the delay in administering the second component should not be such as to lose the beneficial effect of the combination.

Examples of other active ingredients which may be used in such combinations include those mentioned at a) to k) in the paragraph below.

In a further aspect there is provided a pharmaceutical composition (for example, for use as a medicament for the treatment of one of the diseases or conditions listed herein, such as COPD or asthma) comprising a Compound (I) fumarate salt, in particular the Compound (I) hemi-fumarate salt, and at least one active ingredient selected from:
a) a beta-adrenoceptor agonist;
b) a muscarinic receptor antagonist;
c) a joint muscarinic receptor antagonist and beta-adrenoceptor agonist;
d) a toll-like receptor agonist (such as a TLR7 or TLR9 agonist)
e) an adenosine antagonist;
f) a glucocorticoid receptor agonist (steroidal or non-steroidal);
g) a p38 antagonist;
h) an IKK2 antagonist;
i) a PDE4 antagonist;
j) a modulator of chemokine receptor function (such as a CCR1, CCR2B, CCR5, CXCR2 or CXCR3 receptor antagonist); or
k) a CRTh2 antagonist.

### LEGENDS TO FIGURES

FIG. 1 shows an X-ray powder diffraction pattern (XRPD) for 5-((5-methyl-2-((3,4,5-trimethylphenyl)amino)pyrimidin-4-yl)amino)-benzo[d]oxazol-2(3H)-one hemi-fumarate salt.

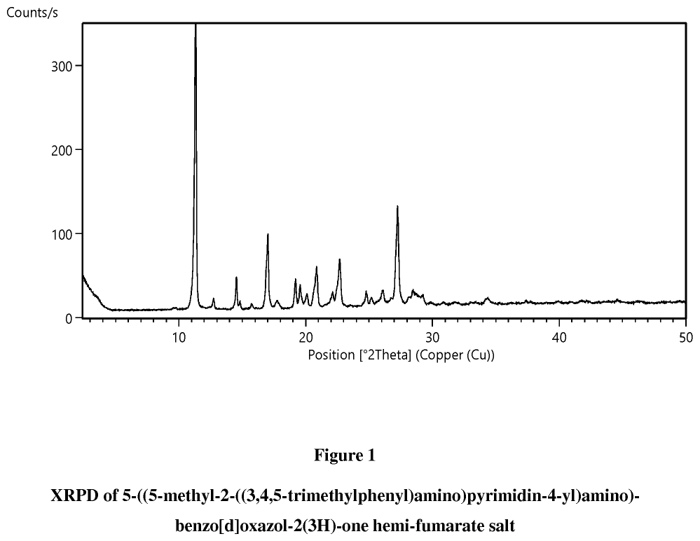

FIG. 2 shows a differential scanning calorimetry trace on 5-((5-methyl-2-((3,4,5-trimethylphenyl)amino)pyrimidin-4-yl)amino)-benzo[d]oxazol-2(3H)-one hemi-fumarate salt. The text on the figure shows the onset temperature of the endotherms.

FIG. 3 shows dissolution profiles for micronized 5-((5-methyl-2-((3,4,5-trimethylphenyl)amino)pyrimidin-4-yl)amino)-benzo[d]oxazol-2(3H)-one hemi-fumarate salt (A), free base (B) and HBr salt (C).

### EXAMPLES

The disclosure is further illustrated by way of the following examples, which are intended to elaborate several embodiments of the disclosure. These Examples are not intended to, nor are they to be construed to, limit the scope of the disclosure. It will be clear that the disclosure may be practised otherwise than as particularly described herein. Numerous modifications and variations of the present disclosure are possible in view of the teachings herein and, therefore, are within the scope of the disclosure.

In the Examples, unless otherwise stated:

(i) yields are given for illustration only and are not necessarily the maximum attainable;
(ii) when given, NMR data is in the form of delta values for major diagnostic protons, given in parts per million (ppm) using perdeuterio dimethyl sulfoxide (DMSO-d₆) as solvent unless otherwise indicated; the following abbreviations have been used: s, singlet; d, doublet; t, triplet; q, quartet; m, multiplet; br, broad;
(iii) chemical symbols have their usual meanings; SI units and symbols are used;
(iv) solvent ratios are given in volume:volume (v/v) terms;
(v) X-Ray Powder Diffraction analysis was carried out as described in the Examples.
(vi) in the Examples given below the number of moles and the yield stated refer to the raw materials and reagents at 100% w/w, thereby taking account of the purity of the materials used.

### Example 1

A solution of fumaric acid (84.9 μL of 80 mM) in MeOH (6.8 μmol) was added to solid Compound (I) free base (5.4 mg, 14 μmol—prepared as described below). The suspension was vigorously stirred for 2 minutes using a vortex stirrer. The suspension thickened and an additional quantity (200 μL) of pure MeOH was added. The suspension was stirred for an additional two hours using a magnetic bar stirrer at ambient temperature. Salt formation and crystallinity was confirmed by X-ray powder diffractometry (see Table 1). The stoichiometry of the salt was determined by NMR.

¹H NMR (600 MHz, DMSO) δ 2.00 (s, 3H), 2.02 (s, 6H), 2.09 (s, 3H), 6.63 (s, 1H), 7.22-7.24 (m, 3H), 7.31-7.32 (m, 2H), 7.85 (s, 1H), 8.34 (s, 1H), 8.77 (s, 1H), 11.60 (s, 1H).

The peak at 6.63 is due to the fumaric acid counter-ion. The integral (1H) shows a stoichiometry of Compound (I):fumaric acid of 1:2, i.e. the hemi-fumarate salt.

For XRPD, samples were mounted on single silicon crystal (SSC) wafer mounts and powder X-ray diffraction was recorded with a Theta-Theta PANalytical X'Pert PRO (wavelength of X-rays 1.5418 Å nickel-filtered Cu radiation, Voltage 45 kV, filament emission 40 mA). Automatic variable divergence and anitscatter slits were used and the samples were rotated during measurement. Samples were scanned from 2-50° 2Theta using a 0.013° step width and a 233 seconds step measurement time using a PIXCEL detector (active length 3.35° 2Theta).

TABLE 1
XRPD Peak positions (°2θ) and intensities

| Position | Intensity |
|---|---|
| 11.28 | vs |
| 12.70 | vw |
| 14.48 | w |
| 14.76 | vw |
| 15.66 | vw |
| 16.92 | m |
| 17.71 | vw |
| 19.14 | w |
| 19.49 | w |
| 20.06 | w |
| 20.82 | m |
| 22.09 | w |
| 22.64 | m |
| 26.05 | w |
| 27.21 | s |
| 28.43 | w |
| 29.20 | w |
| 34.37 | vw |

The following definitions have been used for the relative intensity (%): 81-100%, vs (very strong); 41-80%, str (strong); 21-40%, med (medium); 10-20%, w (weak); 1-9%, vw (very weak).

### Compound (I) Free Base

Compound (I) free base may be obtained as described in WO 2010/085684 or as described in Example 3 below. The Compound (I) free base may be re-crystallised before use as described below.

### Re-Crystallisation of Free Base

DMSO (30 mL, 7.1 mL/g) was added to 5-((5-methyl-2-((3,4,5-trimethylphenyl)-amino)pyrimidin-4-yl)amino)benzo[d]oxazol-2(3H)-one (4.2 g, 11.19 mmol) and the mixture heated to 90° C. Insoluble material was filtered off and, with stirring, heat removed to allow the mixture to come to ambient temperature gradually. The mixture was stirred overnight at ambient temperature and solid material filtered off. The filter cake was washed well with MeOH to yield approximately 2.7 g (64%) of solid after drying under vacuum at ambient temperature.

Alternatively, 5-((5-methyl-2-((3,4,5-trimethylphenyl)-amino)pyrimidin-4-yl)amino)benzo[d]oxazol-2(3H)-one (2.7 g) was dissolved using approximately 24 ml of DMSO at 90° C. MeOH (approximately 5 ml) was added slowly and the mixture brought to ambient temperature slowly. The mixture was stirred overnight at ambient temperature, filtered and the filter cake washed well with MeOH to yield 2.27 g (84%) of solid Compound (I) free base.

### Example 2

Compound (I) (50 mg, 0.13 mmol—prepared as described herein) was dissolved in DMSO (1 mL) at 60° C. under stirring. Fumaric acid (8 mg, 0.7 mmol) was dissolved in EtOH (1 mL) at 60° C. and the resulting solution added dropwise to the Compound (I) DMSO solution at 60° C. No precipitation occurred. The heating was switched off and at approximately 55° C., precipitation started from the solution. The suspension was left to cool to ambient temperature under stirring overnight. The solid was isolated by filtration and the solid form identified by X-ray powder diffractometry.

Thermal events for Compound (I) hemi-fumarate salt were analysed by modulated differential scanning calorimetry (DSC) on a TA DSC Q2000 instrument. 2.7 mg of material contained in a standard aluminium closed cup with a pinhole was measured over the temperature range 20° C. to 380° C. at a constant heating rate of 5° C. per minute, with a overlayed modulation of 0.6° C. at a modulation interval of 45 seconds. A purge gas using nitrogen was used (flow rate 50 mL per minute).

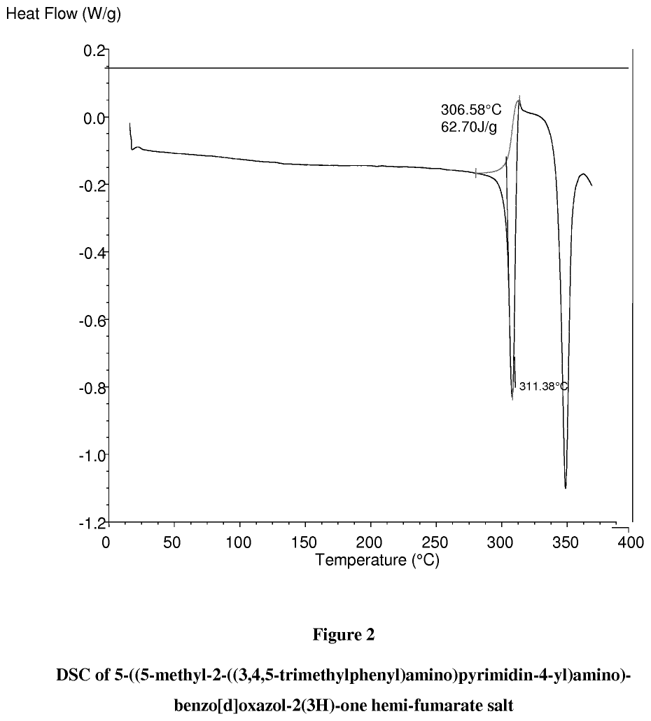

Differential scanning calorimetry on Compound (I) hemi-fumarate salt (FIG. 2) shows an endotherm melting with an onset melting temperature of 307° C.

### Example 3

Compound (I) free base (approximately 1.25 kg—prepared as described below) was dissolved in DMSO (approximately 15.7 L) upon heating to 70-75° C. Fumaric acid (approximately 190 g) was dissolved in DMSO (600 mL) in a separate vessel and was then charged to the solution of Compound (I) free base. A line wash was applied after the solution of fumaric acid went through the transfer line to ensure complete addition of fumaric acid into the solution of Compound (I). Seed crystals of Compound (I) hemi-fumarate salt (prepared, for example, as in Example 2) was charged at a batch temperature of approximately 70-75° C. to initiate crystallization of the salt. Further crystallization was developed by adding approximately 25 L of ethanol over an extended period of time. Subsequently the content of the batch was cooled down in a controlled manner to 5° C. Finally the content of the batch was filtered, washed with ethanol and dried (for example, at 55-60° C. under vacuum).

### Compound (I) Free Base

5-((5-methyl-2-((3,4,5-trimethylphenyl)-amino)pyrimidin-4-yl)amino)benzo[d]oxazol-2(3H)-one (Compound (I)) free base was obtained as described below.

### Example 3-A

2-Chloro-5-methyl-N4-(2-oxo-2,3-dihydro-1,3-benzoxazol-5-yl)-4-pyrimidineamine (94.3 g, 0.34 mol) and 3,4,5-trimethylaniline hydrochloride (69.2 g, 0.40 mol) were suspended in iso-propanol (700 ml) and 2,2,2-trifluoroacetic acid (TFA) (75.5 mL, 0.98 mol). The solution was heated at about 107° C. (external jacket) overnight in a sealed autoclave. After approximately 36 hours some additional TFA (3.8 ml) was charged and the reaction further held at 125° C. (external jacket) under a pressure of about 1.4 bar over at least 66 hours. The resulting reaction mixture was discharged into another reactor. 7N ammonia in methanol (265 ml) and methanol (510 ml) were charged into the reaction mixture, which was then held for at least 2 hours. The content of reactor was then filtered, slurry washed with methanol (1 L) and dried in an oven (damp weight 290.1 g) Analysis of crude solid indicated purity of 73.4%. To enhance the purity the resulting solid was ground down by pestle and mortar, charged in methanol (2 L) in a sonic bath and held for at least 1 hour. The content of the vessel was filtered, and then dried at 50° C. under vacuum to give 5-((5-methyl-2-((3,4,5-trimethylphenyl)-amino)pyrimidin-4-yl)amino)benzo[d]oxazol-2(3H)-one (the purity of which was observed to increase to 80.4%).

In another preparation of 5-((5-methyl-2-((3,4,5-trimethylphenyl)-amino)pyrimidin-4-yl)amino)benzo[d]oxazol-2(3H)-one, 2-chloro-5-methyl-N4-(2-oxo-2,3-dihydro-1,3-benzoxazol-5-yl)-4-pyrimidineamine (94.3 g, 0.34 mol) and 3,4,5-trimethylaniline hydrochloride (69.4 g, 0.40 mol) were suspended in iso-propanol (760 ml) and 2,2,2-trifluoroacetic acid (TFA) (76 mL, 0.98 mol). The solution was heated to 125° C. (external jacket) overnight in a sealed autoclave for at least 72 hours. The resulting reaction mixture was discharged into another reactor. 7N ammonia in methanol (265 ml) and methanol (510 ml) were charged into the reaction mixture, which was held for at least 2 hours followed by 1 hour in a sonic bath. The content of the reactor was then filtered, washed with methanol (3 L), and oven dried at 50° C. 5-((5-methyl-2-((3,4,5-trimethylphenyl)-amino)pyrimidin-4-yl)amino)benzo[d]oxazol-2(3H)-one (362 g, 0.96 mol) and methanol (6.5 L) were charged to a 10 L flask. To the suspension, under nitrogen, was charged benzenesulfonic acid (184.9 g, 1.17 mol). A solution was formed and held for at least 16 hours. The resulting suspension which formed was filtered, washed 5 with methanol (1.0 L) and ethyl acetate (1.0 L), and finally dried to constant weight at 50° C. to give 5-((5-methyl-2-((3,4,5-trimethylphenyl)-amino)pyrimidin-4-yl)amino)benzo[d]oxazol-2(3H)-one benzenesulfonic acid salt.

Finally, 5-((5-methyl-2-((3,4,5-trimethylphenyl)-amino)pyrimidin-4-yl)amino)benzo[d]oxazol-2(3H)-one benzenesulfonic acid salt (396.2 g), ethyl acetate (11.0 L) and 2M sodium hydroxide (2.0 L) were charged to a 20 L flask. Initially a solution was formed, after which solid precipitated. The content of the vessel was held for at least 1 hour, filtered, washed with methanol (˜3 L) and dried to constant weight at 50° C. under vacuum to give 5-((5-methyl-2-((3,4,5-trimethylphenyl)-amino)pyrimidin-4-yl)amino)benzo[d]oxazol-2(3H)-one (Compound (I)) free base.

### Example 3-B

A suspension of 2-chloro-5-methyl-N4-(2-oxo-2,3-dihydro-1,3-benzoxazol-5-yl)-4-pyrimidineamine (1.0 eq.), 3,4,5-trimethylaniline (1.2 eq) and dimethyl sulfoxide (DMSO) (10 rel. vol) was heated to about 85-100° C. for about 16-24 hours. After completion of the reaction (as monitored by HPLC analysis; IPT: <6% of the pyrimidineamine starting material), the mixture was cooled down to about 35° C. Methanol (30 rel vol) was charged, the content of the vessel cooled to 5 to 7° C. and held for about 45-60 min. The resulting solid was filtered off and washed with methanol. The damp solid was charged back to the reactor together with triethylamine (TEA) (2.0 eq) and dimethyl sulfoxide (DMSO) (5 rel vol). The content of the vessel was heated to about 75-80° C. and then cooled back to about 45° C. Methanol (20 rel vol) was charged to the vessel and the content of the vessel held for at least 2-3 hrs. The precipitated solid was filtered off, washed with water and then methanol. The solid was dried in oven at about 55-60° C. under vacuum to give 5-((5-methyl-2-((3,4,5-trimethylphenyl)-amino)pyrimidin-4-yl)amino)benzo[d]oxazol-2(3H)-one (Compound (I)) free base.

### Example 4: Dissolution Measurements

The dissolution profile of different forms of Compound (I) was investigated using a μ-DISS Profiler (pION, MA), a miniaturized dissolution testing apparatus utilizing fiber optic dip-probes connected to a photo diode array detector for scanning absorbance between 200 and 700 nm in situ. In general, 0.5 mg of the micronized material was added to the stirred dissolution media (20 mL 0.1 M acetate buffer, pH 4.5, 200 rpm, at 37° C.). The dissolution profiles were generated by measuring the UV absorbance at 280 nm wavelength. The material of interest was evaluated in triplicate.

Compound (I) free base and Compound (I) hemi-fumarate salt were prepared as described.

Compound (I) HBr salt was prepared as follows:

HBr in MeOH solution (179.8 μL of 80 mM, 14.4 μmol) was added to Compound (I) (5.1 mg, 13.6 μmol). The suspension was vigorously stirred for 2 minutes using a vortex stirrer. The suspension thickened and an additional quantity of pure MeOH (200 μL) was added. The suspension was stirred for an additional two hours using a magnetic bar stirrer at ambient temperature. Salt formation and crystallinity was confirmed by X-ray powder diffractometry.

The particle size of the materials was reduced by micronisation, as follows, using a 2″ Spiral Jet Mill or a 1″ MCOne fluid Jet Mill followed by subsequent particle size distribution (PSD) measurements.

Test substance was fed into the jet mill chamber, via a venturi feed system, by a vibratory feeder. Micronisation was achieved by particle collisions brought about by compressed gas (nitrogen) forced through angled nozzles in the jet mill chamber. Particles of different sizes develop different speeds and momentum and as the particle size is reduced the particles spiral towards the centre of the jet mill and exit via an exhaust into a collection bin. The process parameters that control the particle size, in addition to the inherent properties of the compound to be micronised, are the feed rate, grinding pressure and venturi pressure and these are summarised in Table 2 below.

TABLE 2
Micronisation parameters

| | | | Venturi | Grind | |
|---|---|---|---|---|---|
| | | | Pressure | Pressure | d(0.5) |
| Test Substance | Type of mill | Feed rate | (bar) | (bar) | μm |
| Free base | Spiral Jet | Constant | 3 | 1 | 1.72 |
| | Mill | flow | | | |
| Hemi-fumarate salt | MCOne fluid | Constant | 4 | 2 | 2.20 |
| | Jet Mill | flow | | | |
| Bromide salt | MCOne fluid | Constant | 4 | 2 | 2.52 |
| | Jet Mill | flow | | | |

The PSD was measured using a Malvern Mastersizer 2000 laser diffraction instrument equipped with a Scirocco 2000 dry cell.

Scattering model: Fraunhofer
Analysis model: General purpose (fine)

### Sensitivity: Normal

### Particle RI: 0.0

### Dispersant RI: 1.0

### Absorption: 0.0

Vibration feed rate: 70%
Dispersion pressure: 2.75 bar
Measurement time: 3,105 sec
Measurement snaps: 3105
Background time: 10 sec
Background snaps: 10000

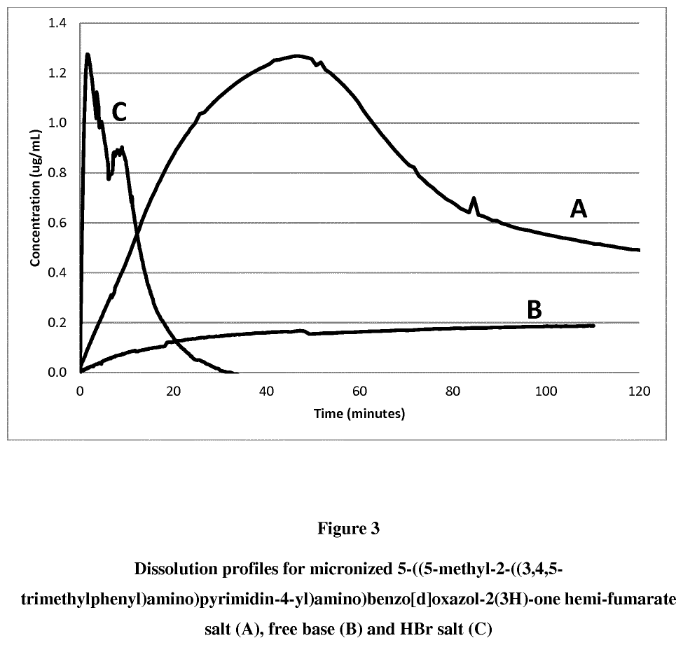

Representative dissolution profiles are depicted in FIG. 3 for micronized free base, micronized HBr salt and micronized hemi-fumarate salt.

The dissolution profile for the hemi-fumarate salt differs significantly from the microsized free base with regards to the initial dissolution rate as well as the measured solubility during this experimental condition. The hemi-fumarate salt shows an enhanced dissolution rate as compared to free base as well as an enhanced solubility (e.g. at 50 minutes, ˜6-fold increase). In addition this enhancement was observed during the duration of the entire experiment. As a comparison, the HBr salt showed a very different dissolution profile not showing any increase in solubility compared to the microsized free base at 1 hour. Both salts depicted in FIG. 3 show altered dissolution profiles compared to the free base. Other salts have also been studied, however only the hemi-fumarate salt produced a suitable dissolution profile. This profile represents an appropriate balance between good (increased) solubility and appropriate kinetics of salt dissociation compared to the free base. Accordingly, material is retained in the lungs only for a suitable time period (aiding safety) and delivers an appropriate concentration of active free base material, so aiding efficacy.

1. A fumarate salt of 5-((5-methyl-2-((3,4,5-trimethylphenyl)amino)pyrimidin-4-yl)amino)-benzo[d]oxazol-2(3H)-one (Compound (I))

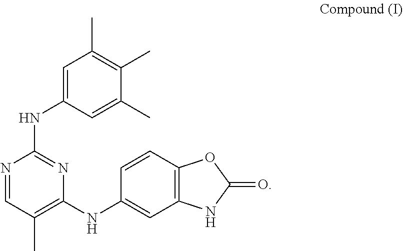 [SMILES: Cc1cnc(Nc2cc(C)c(C)c(C)c2)nc1Nc1ccc2oc(=O)[nH]c2c1]

2. A salt according to claim 1, which is the hemi-fumarate salt of 5-((5-methyl-2-((3,4,5-trimethylphenyl)amino)pyrimidin-4-yl)amino)-benzo[d]oxazol-2(3H)-one.

3. A salt according to claim 2, which is the hemi-fumarate salt of 5-((5-methyl-2-((3,4,5-trimethylphenyl)amino)pyrimidin-4-yl)amino)-benzo[d]oxazol-2(3H)-one in crystalline form.

4. A salt according to claim 3, which is characterised by an X-ray powder diffraction pattern with specific peaks at 11.3, 16.9 and 27.2 °2θ (±0.1°).

5. A salt according to claim 3, which is characterised by an X-ray powder diffraction pattern with specific peaks at about 11.3, 14.5, 16.9, 22.6 and 27.2 °2θ (±0.1°).

6. A salt according to claim 3, which is characterised by a differential scanning calorimetry trace with an endotherm melting with an onset temperature of 307° C.±0.5° C.

7. A process for the preparation of the hemi-fumarate salt of 5-((5-methyl-2-((3,4,5-trimethylphenyl)amino)pyrimidin-4-yl)amino)-benzo[d]oxazol-2(3H)-one according to claim 2, comprising:
(i) Dissolving 5-((5-methyl-2-((3,4,5-trimethylphenyl)amino)pyrimidin-4-yl)amino)-benzo[d]oxazol-2(3H)-one free base in a suitable solvent;
(ii) Dissolving fumaric acid in a suitable solvent;
(iii) Mixing the two solutions;
(iv) Optionally adding seed crystals of the hemi-fumarate salt of 5-((5-methyl-2-((3,4,5-trimethylphenyl)amino)pyrimidin-4-yl)amino)-benzo[d]oxazol-2(3H)-one;
(v) Crystallising the hemi-fumarate salt of 5-((5-methyl-2-((3,4,5-trimethylphenyl)amino)pyrimidin-4-yl)amino)-benzo[d]oxazol-2(3H)-one; and
(vi) Isolating the hemi-fumarate salt of 5-((5-methyl-2-((3,4,5-trimethylphenyl)amino)pyrimidin-4-yl)amino)-benzo[d]oxazol-2(3H)-one.

8. A pharmaceutical composition which comprises a fumarate salt of 5-((5-methyl-2-((3,4,5-trimethylphenyl)amino)pyrimidin-4-yl)amino)-benzo[d]oxazol-2(3H)-one according to claim 1, in association with a pharmaceutically-acceptable excipient, diluent or carrier.

9.-12. (canceled)
